(19) United States
(12) Patent Application Publication
(10) Pub. No.: US 2010/0331978 A1
(43) Pub. Date: Dec. 30, 2010

(54) Antipathogenic Biomedical Implants, Methods and Kits Employing Photocatalytically Active Material

(76) Inventors: Maria Strömme, Uppsala (SE); Ken Welch, Sigtuna (SE); Håkan Engqvist, Osthammar (SE)

Correspondence Address: PORTER WRIGHT MORRIS & ARTHUR, LLP;INTELLECTUAL PROPERTY GROUP, 41 SOUTH HIGH STREET, 28TH FLOOR, COLUMBUS, OH, 43215, US

(21) Appl. No.: 12/918,698

(22) PCT Filed: Feb. 20, 2009
(86) PCT No.: PCT/IB2009/050715

§ 371 Date: Aug. 20, 2010

Related U.S. Application Data
(60) Provisional application No. 61/138,300, filed on Dec. 17, 2008.

(51) Int. Cl. A61F 2/02

(52) U.S. Cl. 623/11.11

(57) ABSTRACT

An antipathogenic biomedical implant is formed throughout its structure of a matrix material comprising at least about 1 weight percent of a photocatalytically active filler which exhibits an antipathogenic effect upon irradiation with light. The photocatalytically active filler is arranged in the matrix material in the implant to receive light irradiated from an external light source. In another embodiment, an antipathogenic biomedical implant comprises at least about 1 weight percent of a photocatalytically active material which exhibits an antipathogenic effect upon irradiation with light, wherein the photocatalytically active material is arranged in the implant to receive light irradiated from an external light source. Methods for providing an antipathogenic biomedical implant, methods for reducing pathogens on a biomedical implant, methods for reducing the bioburden in a biomedical implant installation, and kits for providing an antipathogenic biomedical implant employ the antipathogenic biomedical implants.

### FIELD OF THE INVENTION

The present invention is directed to antipathogenic biomedical implants employing photocatalytically active material which exhibits an antipathogenic effect upon irradiation with light. The invention is further directed to methods and kits for providing antipathogenic biomedical implants and to methods for reducing pathogens on a biomedical implant.

### BACKGROUND OF THE INVENTION

Infections surrounding skin- or bone-penetrating material are a significant health problem for patients and society, with accompanying major treatment costs. Typical treatments of such infections often include massive doses of systemic antibiotics which risk both complications for the patient and development of antibiotic resistant bacteria. The problem of infection is especially pronounced for implants that penetrate the skin, including but not limited to otology implants, catheters, orthopedic implants and dental implants, and for cosmetic materials penetrating the skin, including piercing jewelry and the like. Infection can arise around both permanent implants and temporary implants.

One field that is especially of concern with respect to infection around implants relates to dental materials where caries occur due to bacterial attack. Bacterial attack occurring in an area adjacent or surrounding a dental restoration can result in what are called secondary caries. In this case, the bacterial attack can also occur on the natural tooth, leading to cavity formation in the tooth (caries).

Another area of importance in infection surrounding implants is in the implantation of intraocular lenses (IOL). IOL implantation is often done to replace a natural crystalline lens that is removed due to cataract formation. IOL' s are polymer-based, for example, formed of polymethylmethacrylate (PMMA), silicon-containing polymer, or acrylate polymers as described by Kecova et al, Acta Vet. Brno, 73:85-92 (2004), and typically are a foldable monofocal lens or a multifocal lens. However, IOL implantation is known to involve both infection and inflammation. Another problem associated with IOL is the formation of a secondary cataract. While a secondary cataract can be treated using a YAG laser, implant infections are very difficult to treat.

Methods to reduce the occurrence of infections around implants include administration of systemic antibiotics; local delivery of antibiotics via the implant surface (see Hildebrand et al, “Surface coatings for biological activation and functionalization of medical devices,” Surface & Coatings Technology, 200:6318-6324 (2006); U.S. Pat. No. 7,175,611; Clinical Implant Dentistry and Related Research, 7 (2):105-111 (2005); and U.S. Pat. No. 6,902,397); coating of implants with bactericidal materials, typically platinum, iridium, gold, silver, mercury, copper, iodine, and alloys, compounds and oxides thereof (see U.S. Pat. No. 5,474,797; and Biomaterials, 22(14):2043-2048 (2001)), and photodynamic disinfection via laser irradiation of a toluidine blue compound (see Wilson et al, “Killing of Streptococcus sanguis in biofilms using a light-activated antimicrobial agent,” J Antimicrob Chemother, 37:377-381 (1996)). The release of free radicals to obtain an antibacterial effect via external stimuli has also been proposed (see European Patent No. EP 1 369 137).

These conventional solutions have various problems. As is known, the administration of systemic antibiotics can cause the growing problem of creating antibiotic-resistant bacteria. Additionally, systemically administrated antibiotics affect the whole body and can therefore cause major problems at non-infection sites. In the local delivery of antibiotics via surface coatings, the antibiotics are released over a period of time but infections occurring after the release are not treated with the method and therefore these methods are often followed by systemic administration of antibiotics. Recent research in the coating of implants with bactericidal materials such as silver ions has also found the occurrence of bacteria resistant to the coating materials which leads to great difficulty in treating the infection. Finally, photodynamic disinfection using toluidine blue is effective for one treatment but new addition of toluidine blue needs to be administrated for a subsequent treatment.

Many resin-based dental materials are supplied in an unhardened form and are cured using a light-emitting diode (LED) lamp (see Canadian Patent No. 2,551,089). Unhardened dental materials are often antibacterial (see Orstavik et al, “Antibacterial Activity of Tooth-Colored Dental Restorative Materials,” Dent Res, 57(2):171-174 (1978)), but after hardening, the materials typically do not have an antibacterial effect and secondary caries can easily occur.

Accordingly, there is a continuing need for technologies that reduce or eliminate infections which are encountered with biomedical implants and procedures.

### SUMMARY OF THE INVENTION

It is an object of the present invention to provide improved biomedical implants, methods and kits which overcome various disadvantages of the prior art and provide new means for reducing and/or avoiding infections associated with biomedical implants.

More specifically, in one embodiment, the invention is directed to an antipathogenic biomedical implant which is formed throughout its structure of a matrix material comprising at least about 1 weight percent of a photocatalytically active filler which exhibits an antipathogenic effect upon irradiation with light, wherein the photocatalytically active filler is arranged in the matrix material in the implant to receive light irradiated from an external light source.

In another embodiment, the invention is directed to a method for providing an antipathogenic biomedical implant, which method comprises providing a biomedical implant as described and irradiating the biomedical implant with light of a wavelength and intensity effective to activate the photocatalytically active material.

In an additional embodiment, the invention is directed to a method for reducing pathogens on a biomedical implant, the method comprising installing the biomedical implant in a patient, wherein the implant is installed in a position such that the photocatalytically active filler is arranged to receive light irradiated from an external source, and irradiating the biomedical implant with light of a wavelength and intensity effective to activate the photocatalytically active filler.

In a further embodiment, the invention is directed to a method for reducing pathogens on a biomedical implant, the method comprising irradiating light on a biomedical implant installed in a patient, wherein the biomedical implant is installed in a position such that the photocatalytically active filler is arranged to receive light irradiated from an external source, the irradiated light being of a wavelength and intensity effective to activate the photocatalytically active filler.

In yet a further embodiment, the invention is directed to a method for reducing the bioburden in a biomedical implant installation, comprising irradiating light on the biomedical implant as described with light of a wavelength and intensity effective to activate the photocatalytically active filler, and installing the irradiated implant in a patient.

The present invention is also directed to a kit for providing an antipathogenic biomedical implant. The kit comprises (a) the biomedical implant of claim 1, and (b) a light source operable to emit light of a wavelength and intensity sufficient to cause the photocatalytically active material to exhibit an antipathogenic effect upon irradiation with light from the light source.

In yet another embodiment, the invention is directed to a kit for providing an antipathogenic biomedical implant, comprising (a) a biomedical implant comprising at least about 1 weight percent of a photocatalytically active material which exhibits an antipathogenic effect upon irradiation with light, wherein the photocatalytically active material is arranged in the implant to receive light irradiated from an external light source, and (b) a light source operable to emit light of a wavelength and intensity sufficient to cause the photocatalytically active material to exhibit an antipathogenic effect upon irradiation with light from the light source.

The biomedical implants, methods and kits of the invention are advantageous in providing an antipathogenic effect. Importantly, the lifetime and diffusion distance of hydroxyl radicals formed in the photocatalytic process are extremely short, whereby only the pathogens in the immediate vicinity of the photocatalytically active material will be effected by the process, thereby avoiding damage to adjacent tissue. Thus, the present invention can assist in reducing or eliminating infection at the implant site, without requiring systemic or local administration of antibiotics. Additionally, as will be apparent from the following detailed disclosure, the implants, methods and kits of the invention may be used to provide continuing treatment by successive light irradiation steps, as desired, without additional applications of materials. These and additional objects and advantages of the invention will be more fully apparent in view of the following detailed description.

### DETAILED DESCRIPTION

In a first embodiment, the invention is directed to a biomedical implant. The implant can take any configuration or structure known in the art. In a specific embodiment, the implant is one that is skin penetrating as the present invention is particularly adapted for providing antipathogenic effects to such implants on a continuing basis. The implant can also be bone-penetrating. In one embodiment, the implant is tooth-penetrating.

The antipathogenic biomedical implants of the present invention comprise at least about 1 weight percent of a photocatalytically active material which exhibits an antipathogenic effect upon irradiation with light. Various photocatalytically active materials are known in the art and are suitable for use in the present biomedical implants. In a specific embodiment, the photocatalytically active material comprises one or more of TiO₂, ZnO, ZnS, α-Fe₂O₃, WO₃, SrTiO₃, K₄Nb₆O₁₇, CdS, and oxides with perovskite structure (perovskite oxides). In a more specific embodiment, the photocatalytically active material comprises crystalline titanium dioxide. The crystalline TiO₂ may be of the rutile phase or the anatase phase, or a combination thereof. In one embodiment, the TiO₂ of the rutile phase and/or anatase phase can also include solid solutions of any element beneficial for an antipathogenic or antibiotic effect. Examples of solid solutions include, but are not limited to, one or more of Ca, Sr, Zr, Hf, Mg, ZnSi, P, N and F. As will be described in further detail below, the photocatalytically active material can be used either as a coating on an implant substrate or as a filler material in a matrix material. Further, the photocatalytically active material as a filler in a matrix material may be arranged in a surface layer of the biomedical implant or may be contained throughout the structure of the implant. In a specific embodiment, the implant is formed throughout its structure of a matrix material comprising at least about 1 weight percent of a photocatalytically active filler which exhibits an antipathogenic effect upon irradiation with light, wherein the photocatalytically active filler is arranged in the matrix material in the implant to receive light irradiated from an external light source.

The use of photocatalysts to break down organic compounds in contaminated air and water has been extensively investigated for some time and a number of promising catalysts exist, including TiO₂, ZnO, ZnS, α-Fe₂O₃, WO₃, SrTiO₃, K₄Nb₆O₁₇, CdS, and oxides having a perovskite structure. The most widely used photocatalyst for a number of applications is TiO₂ of anatase phase due to its inertness, corrosive resistance, and inexpensiveness as well as the width and position of its electronic band gap. The width of the band gap of anatase TiO₂ is 3.2 eV, which means that light of a wave length lower than about 385 nm, often referred to as UV-A or black light, is needed to form electron-hole pairs in the material. Furthermore, the upper edge position of the valence band is positioned low enough on an energy scale for the holes which are produced when the material is illuminated with light of the proper wave length to react with water and produce highly reactive hydroxyl radicals (OH). Both the holes and the hydroxyl radicals are very strong oxidants which can be used to oxidize many organic compounds. The position of the conduction band of anatase TiO₂ is simultaneously high enough to drive the reaction involving electrolytic reduction of molecular oxygen (O₂) to superoxide (O₂⁻). It has been found that superoxide is almost as important as the holes and the hydroxyl radicals in breaking down organic compounds (see Fujishima et al, TiO₂ Photocatalysis, Fundamentals and Applications, BKC Inc, Tokyo (1999)). Thus, for photocatalytic reactions involving anatase TiO₂ to be as efficient as possible, both water and oxygen should be present. TiO₂ of rutile phase is also photocatalytically active. The valence band of this material is positioned very close to the valence band of anatase but the conduction band is about 0.2 eV lower in energy than that of the anatase material, meaning that the driving force for superoxide formation is not as strong as for the anatase phase.

Crystalline titanium dioxide is known to be bactericidal under the illumination of UV light (see Ibáñez et al., Journal of Photochemistry and Photobiology A: Chemistry, 157(1):81-85 (2003). Surfaces coated with titanium dioxide show antibacterial and self-cleaning characteristics related to the photocatalytic properties of titanium dioxide in the anatase form (see Pelizzetti et al, Nouv. J. Chim., 8:547-550 (1984); Pelizzetti et al, Heterogeneous Photocatalysis, J. Wiley and Sons (1989); Pelizzetti et al, Adv. Colloid and Interf. Sci., 32:271-316 (1990); Ollis et al, Photocatalytic Purification and Treatment of Water and Air, Elsevier, Amsterdam (1993); Pelizzetti et al, “Mechanism of the photooxidative degradation of organic pollutants over titanium dioxide particles,” Electrochim. Acta, 38:47-55 (1993); and WO 2006/043166). It is also known that materials with photocatalytic properties denature bacteria as shown in the Escherichia Coli case study by Sunada et al, “Bactericidal and Detoxification Effects of TiO₂ Film Photocatalysts,” Environ. Sci. Technol., 32:726 (1998).

In addition to applications in water and air cleaning, the photocatalytic activity of TiO₂ and other materials has been suggested for killing of cancer cells (see Blake et al, “Application of the photocatalytic chemistry of titanium dioxide to disinfection and the killing of cancer cells,” Sep. Purif. Methods, 28:1-50 (1999), and Seo et al, “Development of water-soluble single crystalline TiO₂ nanoparticles for photocatalytic cancer-cell treatment,” Small, 3:850-853 (2007)), and disinfection of walls and floors in operating rooms, self-cleaning windows, etc. (see Fujishima et al, supra). Photocatalytic killing of bacteria, viruses and fungi has also been studied (see Hajkova et al, “Photocatalytic effect of TiO₂ films on viruses and bacteria,” Plasma Process. Polym., 4:5397-5401 (2007), and references therein) and attempts to explain the mechanism of the killing process have been made (see Maness et al, “Bactericidal activity of photocatalytic TiO₂ reaction: toward an understanding of its killing mechanism,” Applied and Environmental Microbiology, 65:4094-4098 (1999); Blake et al, supra). Preclinical trials have been carried out involving injection of photocatalytic TiO₂ particles into tumors in mice with subsequent irradiation with UV light (see Cai et al, Cancer Research, 52:2346 (1992). Treatment of acne, skin lesions, wounds, burns, etc., on skin by applying a topical composition containing a photocatalyst-containing composition and letting ordinary daylight or a lamp activate a photocatalytic process that kills the microorganisms involved in the skin problems has also been described (see WO/2004/064881 (Skin treatment formulations); Thai Application No. 0501003619 (An antimicrobial composition for topical application and a method thereof); JP 11-005729 (Cosmetic having photocatalytic function); and JP 2006-056825 (Skin care preparation and face mask using the same)). Lamp systems for activating photocatalytic processes on skin have also been described (JP 2006-068420 (Ultraviolet sterilizer)).

The implants according to the present invention comprise the photocatalytically active material in an amount of at least about 1 weight percent (wt. %). In a specific embodiment, the implant comprises the photocatalytically active material in an amount greater than about 5 wt. %, and in a more specific embodiment, the implant comprises the photocatalytically active material in an amount greater than about 10 wt. %. In an additional embodiment, the photocatalytically active material has a crystalline grain structure, and the grain size is less than about 1 mm, or, more specifically, less than about 100 micrometer (μm). In an even more specific embodiment, the grain size is less than about 10 micrometer, or less than about 1 micrometer. Further, in more specific embodiments, the grain size is greater than about 1 nm or greater than about 5 nm. To increase the efficiency of the photocatalysis, a photocatalytically active material of large surface area is beneficial. A large surface area is ensured by using small particle sizes of the photocatalytically active material. For example, in specific embodiments, the surface area of the photocatalytically active material is greater than about 0.1 m²/g, greater than about 10 m²/g, greater than about 30 m²/g, and greater than about 50 m²/g, respectively.

The implant may include additives to enhance the photocatalytic activity. Non-limiting examples of other components that may be present include sodium perborate, magnesium silicate, and citric acid. These ingredients particularly serve to enhance the photocatalytic activity of photocatalytically active material such as titanium dioxide when the material is exposed to light.

In one specific embodiment, the photocatalytically active material comprises TiO₂ nanoparticles with a major portion of the particles, i.e. greater than about 50% by weight, being below about 100 nm in size and wherein a majority, i.e. greater than about 50% by weight, of the particles which are less than 100 nm in size are of the anatase phase. In accordance with techniques known in the art, such TiO₂ nanoparticles may be produced by sol-gel, solid state diffusion or molecular assembly techniques.

In another embodiment, the photocatalytically active material may comprise porous particles, thus providing an increased surface area for the photocatalytic process. In specific embodiments, the pore size (open porosity) in the particles is within a range of about 0.1 nm to about 10 micrometer, or within a range of about 0.1 nm to 100 nm. In yet another embodiment, the photocatalytically active material comprises porous TiO₂ particles or TiO₂ nanotubes. In the case of TiO₂ nanotubes, the above mentioned sizes apply to the diameter of the tubes, and the length of the tubes can be up to several micrometers.

According to one embodiment of the invention, photocatalytically active material is employed as a filler in a matrix of an implant biomaterial. The filled matrix material may be arranged at a surface of the implant structure or may be employed throughout the implant structure. In a specific embodiment, the matrix material is employed throughout the structure of the implant and the photocatalytically active filler is arranged in the matrix in at least a surface layer of the biomedical implant. In another embodiment, the photocatalytically active filler is arranged in the matrix throughout the structure of the biomedical implant. The filler particles can be porous as discussed above or non-porous and of any shape, including powders, granules or the like.

In one specific embodiment, the filler is added to an unhardened biomaterial such as an injectable in-situ-setting or hardening polymer. Such polymers are known in the art and include, but are not limited to, polyurethane, silicone polymers, polyethylene, bisphenol-A diglycidylether methacrylate (in situ-hardening), polymethylmethacrylate, and glass polyalkenoate cements, optionally in combination with X-ray opacity additives such as barium sulfate and zirconium dioxide. In a specific embodiment, the matrix comprises an in situ-hardening polymer such as bisphenol-A diglycidylether methacrylate (Bis-GMA) or glass polyalkenoate cement, or a combination thereof. The Bis-GMA and glass polyalkenoate cement materials may also contain additives suitable for individual implant use, for example, color additives, X-ray opacity additives, and/or photoinitiators for implants used as dental material. The photocatalytically active material can also be added as a filler to ceramic matrix material, for example to calcium phosphate or calcium sulphate, or to calcium aluminate injectable biomaterials, e.g. monocalcium aluminate, in combination with X-ray opacity additives. The photocatalytically active filler can also be added to combinations of injectable polymers and ceramics, e.g. combinations of calcium aluminate and glass ionomer cements as well.

In a specific embodiment, the photocatalytically active filler comprises crystalline titanium dioxide filler particles. Such particles are commercially available from powder manufacturers or companies, for example, Sigma Aldrich, Degussa and Strem. In a more specific embodiment, the photocatalytically active material comprises TiO₂ nanoparticles of anatase phase. These particles may be made by e.g. sol-gel techniques, or they may be purchased from a nanoparticle manufacturer. The TiO₂ nanoparticles P25 produced by Degussa Chemical Company (Germany) consist of about 75% anatase phase and 25% rutile phase. These particles are one non-limiting example of a starting material for carrying out the present invention. The pathogenicidal activity of TiO₂ to longer wavelengths (visible light) may be obtained via the addition of e.g. solid solutions into the crystalline phases, examples of solid solutions including nitrogen ions. In a specific embodiment, a photocatalytically active material as described is employed as a filler material for an implant for use in dentistry, wherein the implant may be in the form of e.g. restoratives, temporary fillings, cements, adhesives, base and liners. Yet another specific area for employing a photocatalytically active material as described is as a filler material for an implant in orthopedics.

According to yet another embodiment of the invention, the photocatalytically active material can be added to a resorbable carrier material, examples of which include, but are not limited to, ceramics, polymers, including hydrogels, or combinations thereof, and the like. In a specific embodiment, the photocatalytically active material which is added to the resorbable material is titanium dioxide filler material. Preferably, for a ceramic carrier, e.g., calcium phosphate cements, calcium sulphate cements, calcium silicate cements, and combinations thereof, the material is delivered in the form of a powder and a liquid where the powder and the liquid are mixed to a paste and injected via a syringe to the tissue. Typical descriptions of resorbable injectable material systems which are suitable for use in combination with a photocatalytically active material according to the invention are described by Bohner et al, Biomaterials, 26:6423-6429 (2005), incorporated herein by reference. Non-injectable ceramic biomaterials especially include calcium phosphate materials. For hydrogel carriers such as, e.g., hyaluronic acid, the powder and hydrogel can be premixed in a syringe ready for injection with no extra on site mixing needed. As a non-limiting example, a photocatalytically active material such as titanium dioxide in a resorbable carrier material is suitably used in periodontology. The material is also suitable for use in shallow caries lesions to aid with antibacterial and biomineralization of the shallow caries lesion.

While titanium dioxide in low concentration has been used in dental materials for a whitening effect and to reduce the translucence of the material, in such cases the amount of added titanium dioxide is typically below 3 wt. % and generally the crystallinity of the oxide is not of concern or specified. As will be apparent from the Examples set forth below, photocatalytically active titanium dioxide has a crystalline structure.

Other components in the implant material in the vicinity of the photocatalytically active material should have a high transparency to the light used for photocatalytic activation. When TiO₂ particles constitute the photocatalytically active material, the components should be transparent to UV-A light, i.e. light with a wave length between 400 and 315 nm.

According to another embodiment of the invention, the photocatalytically active material is deposited as a coating on an implant substrate. Non-limiting examples of implant substrate materials include titanium, stainless steel, cobalt chromium alloys, tantalum, polyurethane, silicon, polyethylene, aluminum oxide, zirconium dioxide, hydroxymethylmethacrylate, polymethylmethacrylate, and the like. In a specific embodiment, the photocatalytically active material is provided as a coating on an implant substrate comprising titanium, stainless steel, polyurethane, silicon, a methacrylate or polyethylene. In a more specific embodiment, the implant substrate comprises titanium. Titanium is widely used as a biomedical implant, e.g. in orthopedics and dentistry, as it is biocompatible and integrates well with tissue. These properties result to a major degree from the titanium dioxide that forms naturally on the surface. The bioactivity of the crystalline phases of titanium dioxide have been documented (see Kokubo et al, “Titania-based bioactive materials,” Journal of the European Ceramic Society, 27:1553-1558 (2007)). Bioactivity in this context is defined as the ability to form a chemical bond to bone. Bioactivity of crystalline titanium dioxide has been described in thin coatings and in self-setting PMMA containing more than 50 wt. % fine grained powder.

In one embodiment, the photocatalytically active material is deposited on the implant substrate with a coating thickness suitably above about 5 nm and below about 1 mm, and in a specific embodiment, the coating thickness is less than about 100 micrometer. The coating can be deposited using any deposition method, and preferable, the deposition method employs a maximum temperature below 800° C., and preferably employs a maximum temperature below about 400° C. Non-limiting examples of deposition methods include sol-gel methods, physical vapor deposition (including sputtering, arc evaporation and cathodic evaporation), and chemical vapor deposition.

In one embodiment, the implant comprises a surgical implant substrate and a thin film coating deposited on the substrate, the thin film coating comprising TiO₂₋ₓM_{y}, wherein M is one or more elements which does not adversely effect adherence of the coating to the substrate, y is the sum of the mols of all M elements, 0≦x<2 and 0≦y≦1, and wherein an outermost portion of the thin film coating is crystalline with crystalline grains larger than 1 nm. In yet a further embodiment, a method of forming the implant comprises cleaning and sputter etching a surface of a metallic substrate to remove native oxide, and depositing a thin film coating on the substrate surface, the thin film coating comprising TiO₂₋ₓM_{y}, wherein M is one or more elements which does not adversely effect adherence of the coating to the substrate, y is the sum of the mols of all M elements, 0≦x<2 and 0≦y≦1, and wherein an outermost portion of the thin film coating is crystalline with crystalline grains larger than 1 nm. When the thin film coating includes a gradient composition, the gradient composition may comprise from 99% to 0.01% of the thin film coating thickness. In a more specific embodiment, the gradient composition may comprise less than about 90% of the thin film coating thickness. In a further embodiment, the gradient composition comprises at least about 10% of the thin film coating thickness. In a specific embodiment, the gradient composition has a thickness of greater than about 7 nm, more specifically greater than about 15 nm, and even more specifically greater than about 40 nm, and/or a thickness less than about 30 micrometers, more specifically less than about 1 micrometer, and even more specifically less than about 200 nm. In a further specific embodiment, the gradient composition has a thickness of from about 40 nm to 200 nm.

In a specific embodiment, to increase the crystallinity of the coating, for example to increase the crystalline grain size, the coating is heat-treated post deposition. The heat treatment is made at a temperature low enough to not compromise the mechanical stability of the substrate. For Ti containing substrates, this temperature is suitably below 500° C., more specifically below 450° C., and in a further embodiment, below 400° C. For substrates mainly containing Co—Cr, this temperature is suitably below 900° C., preferably below 600° C., and, in a specific embodiment, below 500° C. For substrates mainly containing stainless steel, this temperature is suitably below 600° C., preferably below 500° C., and, in a specific embodiment, below 400° C. The heat treatment is typically performed for less than 24 hours, more typically for less than 10 hours, even more typically for less than about 2 hours. The heat treatment can be done in the presence of any gas that does not adversely affect the functionality of the coating. Examples of such gases include, but are not limited to, air, oxygen, nitrogen, argon, helium, and krypton and any mixture thereof. One of ordinary skill in the art will appreciate the appropriate temperature intervals suitable for other types of substrates.

In a specific embodiment, to increase the coating adhesion, the substrate is first cleaned using conventional cleaning procedures before conducting the deposition process for forming the thin film coating. Further, the substrate may also be sputter-etched before depositing the thin film coating, for example, in order to remove native oxide on a metal implant substrate. Typically, such sputter-etching is not employed in the manufacture of composite materials using ceramic and/or polymeric implants substrates.

If desired, the coating may be porous, and in one embodiment, may be nanoporous, having pores of a size in the range of about 0.1-100 nm. Porosity of the coating may be controlled via the deposition process for example, by temperature and, when using sputtering, the partial pressures of argon and oxygen, mainly that of the argon pressure. For titanium substrate implants, one option according to the invention is to heat treat the native titanium dioxide, i.e. with no addition of a coating step, at temperatures below 600° C., or, more specifically, below 400° C., to obtain a surface layer with a crystallinity above about 1 wt. % rutile or anatase or combinations thereof. For Ti implants, surface treatments including anodic oxidation as described in Dental Materials, 25(1):80-86 (January 2009) may be employed to obtain a crystalline oxide surface suitable as the desired surface coating composition.

Non-limiting examples of implants including the photocatalytically active material as a coating include: orthopedic implants, otology implants, dental implants, catheters, piercing implements and jewelry, and intraocular lenses. According to yet another embodiment of the invention, the photocatalytically active material as a coating, for example as a crystalline titanium dioxide coating, can be applied to the implant in an area of the implant adapted to penetrate the skin. This means that the coating is applied in the area where risk of bacterial infection and pathogens is highest. Such implants include, but are not limited to orthopedic fracture fixation devices, prosthetic dentistry, IOLs and catheters.

Crystalline titanium dioxide is known to be bone bioactive, meaning that it has the capacity to form a bond to bone. According to yet another embodiment of the invention, this effect can be combined with the antipathogenic effect for implants in hard tissue applications, where otology, dentistry, prosthetic dentistry and orthopedics can be mentioned as non-limiting examples.

Non-limiting examples of pathogens which can be reduced or eliminated with the use of a biomedical implant as described and light activation of the photocatalytically active material include, but are not limited to, Pityrosporum, Mallassezia, Coryneform, Propionibacterium, Micrococcus, Staphylococcus, Proteus and Trichophyton. Specific examples are: Pityrosporum ovate and Malassezia furfur and other microorganisms which occur in the hair or on the scalp, Coryneform bacteria and other microorganisms typically present in the underarm area; Propionibacterium acnes, Micrococcus species, Staphylococcus aureus and other microorganisms present on and responsible for lesions of the skin; and Staphylococcus epidermidis, Proteus vulgaris and Trichophyton mentagrophytes and other microorganisms typically present on the feet, the HSV virus, such as herpes simplex, the human papilloma virus (HPV) as well as microorganisms listed in Table IV and mentioned in the main text in of Blake et al, “Application of the photocatalytic chemistry of titanium dioxide to disinfection and killing of cancer cells,” Separation and Purification Methods, 28:1-50 (1999) which is hereby incorporated by reference.

In accordance with the present methods, the photocatalytically active material is arranged in the biomedical implant to receive light irradiated from an external light source, i.e., external to the implant, and the method comprises irradiating the biomedical implant with light of a wavelength and intensity effective to activate the photocatalytically active material. The light source for illumination of the photocatalytically active material to obtain an antipathogenic effect can be stationary, portable, handheld, or in any other configuration. In a specific embodiment, the light source is handheld. Exemplary light sources include, but are not limited to, an incandescent lamp, a gas discharge lamp, a halogen lamp, a fluorescent lamp, a laser, a light-emitting diode, or any combinations thereof.

One skilled in the art will be able to determine the appropriate wavelength and intensity necessary to obtain an antipathogenic effect according to the invention, depending on the photocatalytically active material. In a specific embodiment, wherein the photocatalytically active material comprises titanium dioxide, the light-emitting source provides light in the UV range, preferably mostly in the range of 200 nm to 600 nm. In a specific embodiment, the light-emitting source provides light in the range of 300 nm to 450 nm. The intensity from the light source is typically below 1000 W cm ⁻². In specific embodiments, the intensity of light is below 200 W cm⁻², or below 100 W cm⁻². The intensity reaching the area to be treated, i.e., activated, is typically above 0.1 mW cm⁻², and, in more specific embodiments, is above 0.5 mW cm⁻², above 1 mW cm⁻², or above 5 mW cm⁻². For comparison, it may be noted that the UV light emitted from the sun and reaching the surface of the earth is about 1 mW cm⁻², while the total effect emitted from a normal incandescent lamp is about 0.07 microW. The illumination dose needed for efficient treatment is strongly dependent on the photocatalytic efficiency of the active material. The stronger photocatalyst used and the larger its total surface area, the lower the dose of illumination with photons in the proper wavelength range will be needed. The dose needed for efficient treatment is normally above 0.01 mJ cm⁻², typically above 0.1 mJ cm⁻², more typically above 0.5 mJ cm ⁻², even more typically above 1 mJ cm ⁻², most typically above 5 mJ cm ⁻².

Optionally, the implants may be irradiated prior to their installation in a patient to reduce the bioburden encountered in the implant installation. In this embodiment, a biomedical implant, for example dental implants or endosseous implants in general, before surgical utilization can be irradiated with light of a wavelength and intensity effective to activate the photocatalytically active filler. The thus irradiated implant can then be installed in a patient with a reduced bioburden and therefore a reduced risk of infection. In one embodiment, an implant is immersed in water, preferentially de-ionized, followed by ultraviolet light irradiation (230-380 nm, preferentially 250-320 nm) before use. Additionally, UV irradiation on a fully anatase coated surface, for example, which shows elevated photocatalytic activity, will result in a modification of its surface status conferring a super-hydrophilic property, which results in a substantial increase in wettability of the surface water itself (the contact angle with the film gradually decreases to 0 degrees) and can foster biocompatibility. Both positive properties can be obtained under ultraviolet light irradiation before use.

The biomedical implants of the invention may be of any type and structure. Non-limiting examples of implants comprising coatings or filler particles of the photocatalytically active antipathogenic material according to the invention in the form of catheters include such for draining urine from the urinary bladder as in urinary catheterization, e.g., the Foley catheter or suprapubic catheterization, drainage of urine from the kidney pelvis by percutaneous nephrostomy, drainage of fluid collections, e.g. an abdominal abscess, administration of intravenous fluids, medication or parenteral nutrition, angioplasty, angiography, balloon septostomy, balloon sinuplasty, direct measurement of blood pressure in an artery or vein, direct measurement of intracranial pressure administration of anesthetic medication into the epidural space, the subarachnoid space, or around a major nerve bundle such as the brachial plexus, subcutaneous administration of insulin or other medications, with the use of an infusion set and insulin pump, central venous catheter as a conduit for giving drugs or fluids into a large-bore catheter positioned, for example, either in a vein near the heart or just inside the atrium, or a Swan-Ganz catheter. Yet another example of the present invention comprises a photocatalytically active antipathogenic material coating on Hoffman instruments.

Non-limiting examples of the use of coatings or filler particles of the photocatalytically active antipathogenic material in an implant according to the invention in dentistry include: in situ hardening materials, periodontology, treatment of caries lesions and dental implants. In a specific embodiment, the photocatalytically active material is also bone-bioactive. Non-limiting examples of the use of coatings or filler particles of the photocatalytically active antipathogenic material in an implant according to the invention in orthopedics include, e.g., fracture fixation, spine devices, and prostheses, and craniomaxilliofacial devices. Again, the photocatalytically active material may be bone-bioactive in specific embodiments. Bone-bioactive material is also advantageous for otology implants as well. Non-limiting examples of the photocatalytically active antipathogenic material containing implants also include devices used in piercing applications, including jewelry, and cataract surgery IOL materials as described in e.g. Kecova et al, supra. In the case of IOL's, the antipathogenic effect is present during exposure to daylight given that the coating is on the IOL-implanted lens in the eye, providing extra benefit for the application in reducing the incidence of infections and also secondary cataract. In a specific embodiment, the thickness of a coating on an IOL is below about 400 nm, and in more specific embodiments, is below 100 nm or below 20 nm. The thin coatings can reduce the effect from differences in refractive index between the coating material and the lens material (polymer).

According to yet another embodiment, the invention is directed to a kit comprising the biomedical implant according to the present invention and a light source operable to emit light of a wavelength and intensity sufficient to cause the photocatalytically active material to exhibit an antipathogenic effect upon irradiation with light from the light source. The implant composition as well as the parameters of the light source may vary depending on the infection or disease to be treated or prevented. The light source should emit light within a wavelength region that stimulates the photocatalytic effect of the material. The maximum wavelength required to activate the photocatalytic process depends on the electronic bandgap of the photocatalytically active material used and may be determined by one skilled in the art. For an active material which mainly comprises TiO₂ nanoparticles of anatase phase, the light source should emit photons with a wavelength in the UV-A region; i.e. in the range of from 400 nm to 315 nm, preferably in the range of from 385 nm to 315 nm. To reduce the risk of side effects and damage to healthy cells, the light should be filtered so that photons of wavelength lower than 315 nm do not reach the treated area. Preferably, the emitted spectrum of the light source should be designed to emit photons of wavelength larger than 320 nm or alternatively the light below this wave length should be filtered. For anatase nanoparticles, the optimal wavelength region is below 385 nm to induce an optimum photocatalytic effect and above 320 nm to minimize side effects and damage to healthy mammalian cells.

A method according to the invention therefore comprises, in an exemplary embodiment, a first step wherein the biomedical implant is placed in site. A biomedical implant containing crystalline titanium dioxide acts as a bone bioactive material in contact with body fluids. The method includes a second step wherein the material is illuminated using the light-emitting device to obtain an antipathogenic effect at the surface of the material. The second step can be repeated several times at different time points as desired. Surprisingly, the combination of a photocatalytically active material and illumination with the described light results in an antipathogenic system and a method for prevention and treatment of infections. For hard tissue applications (bone and teeth), the combination surprisingly also provides bone bioactivity.

Various embodiments of the invention are demonstrated by the following examples.

### EXAMPLE 1

A series of experiments were performed to deposit titanium dioxide coatings on metallic substrates. Specifically, graded titanium dioxide thin films were prepared in a reactive DC magnetron sputtering unit (Balzers 640R). The sample holder was rotated and a pure titanium target (99.9%) was used for depositing a thin film layer. Pure argon (99.997%) and oxygen (99.997%) were used for the reactive sputtering. The magnetron effect and oxygen partial pressure were chosen to 1.5 kW and 1.5 10⁻³ mbar, respectively.

In a first experiment (Experiment 1), a first set of samples were deposited by first depositing a layer of pure titanium of 50 nm thickness. On the surface of this pure titanium layer, a second layer of 50 nm was formed with the oxygen flow gradually increasing from near zero to a constant value to give an oxygen content gradient in the resulting Ti oxide layer. When the oxygen flow was high enough to produce TiO₂, the flow was held constant at this flow to form a 100 nm thick TiO₂ layer. The substrate temperature during these steps was held constant at 350° C. The resulting material is referred to as Sample 1. A second experiment (Experiment 2) was conducted by repeating the procedure in Experiment 1 and by additionally heat treating the sample for 1 hour in air at 390° C. post deposition. The resulting material is referred to as Sample 2. A third experiment (Experiment 3) was conducted by depositing the titanium dioxide coating without substrate heating and no heat treatment post deposition. The resulting material is referred to as Sample 3. A fourth experiment (Experiment 4) was conducted by repeating the procedure in the third experiment and by additionally heat treating the sample for 1 hour in air at 390° C. post deposition. The resulting material is referred to as Sample 4.

The obtained coatings were characterized using X-ray diffraction (XRD) for phase composition (detection of crystalline phases), scanning electron microscopy (SEM) for studying the film thickness in cross-section (LEO 440), and X-ray photoelectron spectroscopy (XPS) for detecting the gradient structure. The coating adhesion was measured using Rockwell C indentation.

The outermost regions of the coatings of Samples 1 and 2, deposited at 350° C., were nanocrystalline. The crystallinity of Sample 2, produced with the post deposition heat treatment, was higher than that of Sample 1. Sample 1 contained anatase phase TiO₂ with a grain size according to Scherrer's equation of about 15 nm while Sample 2 contained mainly anatase phase TiO₂ with a grain size according to Scherrer's equation of about 35 nm.

Analyzing Samples 3 and 4 using XRD showed that the coating deposited with no substrate heating and without post deposition treatment (Sample 3) was amorphous and that the sample deposited without substrate heating but with post deposition heating (Sample 4) was crystalline. XPS analysis of the gradient coatings showed that the gradient zones in the coatings were about 50 nm as is evident from XPS depth profiles. Substrate adhesion testing using Rockwell C indentation showed that the adhesion was the highest for the gradient coatings deposited using substrate heating, Samples 1 and 2. The heat treatment of Sample 2 did not seem to affect the adherence as no difference could be found between Samples 1 and 2 in the adhesion testing.

These experiments show that the titanium oxide-containing coatings having a gradient in the oxygen content and deposited at 350° C. (Samples 1 and 2) were nanocrystalline and had a high adhesion. They also show that samples that are sputtered with substrate heating and thereafter heat treated (Sample 2) have higher crystallinity than those that are not heat treated (Sample 1). They further show that samples that are amorphous after sputter deposition at room temperature (Sample 3) can be made crystalline by only moderate heat treatment (Sample 4).

Samples 1-4 were tested for their antibacterial effect under UV light. A glass substrate was used for comparison. 10 microliters of bacteria (Staphylococcus epidermidis) solution grown in liquid culture was applied to samples 1-4 and the glass substrate prior to UV illumination (intensity 2 mW cm⁻², peak intensity of light at 365 nm). Samples 1-4 and the glass substrate were illuminated for 1 hour, and then the bacteria were collected from the surfaces by pressing the surfaces into an agar gel culture medium. The culture media were incubated at 37° C. for 24 hours to assess bacteria viability. While the glass substrate showed no antibacterial effect, Samples 1, 2 and 4 showed very good antibacterial properties. On these samples, few to no colony forming units (cfu) were observed on the culture media. Sample 3 showed lower antibacterial effect as compared to Samples 1, 2 and 4, but enhanced effect compared to the glass substrate. As an additional control, the experiment was repeated with Samples 1-4 and the glass substrate without UV irradiation. This test showed no antibacterial effect on any test surface.

### EXAMPLE 2

A series of experiments were performed to test the bioactivity and antibacterial effect of crystalline titanium dioxide as a photocatalytically active material in accordance with the invention. Specifically, anatase titanium dioxide was coated on a titanium implant and a polyurethane catheter. Uncoated implants were used as references. The coatings were achieved using a sol-gel technique as described by Rossi et al, Journal of Biomedical Materials Research Part A, 82A(4):965-974 (2006), incorporated herein by reference.

Additionally, an implant material was made by blending 90 wt. % pre-cured resin-based dental adhesive and 10 wt. % TiO₂ grains (mixture of 25 nm anatase and rutile grains, P25 Degussa). A resin-based dental adhesive with dental glass as filler was used as a comparative material. Thin layers (approx. 100 micrometer) of the materials were hardened using a blue LED curing light (Ivoclar) according to the manufacturer's instructions. After hardening, the materials were polished with 1000 grit sandpaper. The materials were stored in sterile water for 24 h before testing.

Bioactivity was evaluated according to the method as described in Kokubo et al, Biomaterials, 27:2907-2915 (2006). The antibacterial effect was evaluated using the method as described in Orstavik et al, “Antibacterial Activity of Tooth-Colored Dental Restorative Materials,” Dent Res, 57(2):171-174 (1978), with the addition that all materials were illuminated with a 1 mW cm⁻² UV (peak intensity at 365 nm) light source when in contact with the bacteria.

The results showed that the implants containing crystalline titanium dioxide in the surface (both as filler and as coating) were bioactive and had a pronounced antibacterial effect. The reference materials were inert and showed no antibacterial effect.

### EXAMPLE 3

A paste material for treatment of shallow caries lesions comprised 10 wt. % titanium dioxide (grain size of 25 nm, mixture of anatase and rutile grains from Degussa P25) blended in a hyaluronan gel. The gel was applied to shallow caries lesions and illuminated with UV light (intensity 5 mW cm⁻², peak intensity at 365 nm). After illuminating and rinsing, viable bacteria levels in the lesion were reduced or completely removed.

The specific illustrations and embodiments described herein are exemplary only in nature and are not intended to be limiting of the invention defined by the claims. Further embodiments and examples will be apparent to one of ordinary skill in the art in view of this specification and are within the scope of the claimed invention.

1. An antipathogenic biomedical implant, formed throughout its structure of a matrix material comprising at least about 1 weight percent of a photocatalytically active filler which exhibits an antipathogenic effect upon irradiation with light, wherein the photocatalytically active filler is arranged in the matrix material in the implant to receive light irradiated from an external light source.

2. (canceled)

3. The biomedical implant of claim 1, wherein the photocatalytically active filler is arranged in the matrix in at least a surface layer of the biomedical implant.

4. The biomedical implant of claim 1, wherein the matrix material comprises ceramic, polymer, or a mixture thereof.

5. The biomedical implant of claim 4, wherein the matrix material comprises one or more of calcium phosphate, calcium sulphate, calcium aluminate, calcium silicate, polyurethane, silicone polymer, polyethylene, bisphenol-A diglycidylether methacrylate, and glass polyalkenoate.

6. The biomedical implant of claim 1, wherein the photocatalytically active filler comprises one or more of TiO₂, ZnO, ZnS, α-Fe₂O₃, WO₃, SrTiO₃, K₄Nb₆O₁₇, CdS, and perovskite oxide.

7.-8. (canceled)

9. The biomedical implant of claim 6, wherein the photocatalytically active filler is crystalline and has a grain size less than about 1 mm, less than about 100 μm, less than about 10 μm, or less than about 1 μm, and greater than about 1 nm or greater than about 5 nm.

10. The biomedical implant of claim 6, wherein the photocatalytically active filler has a surface area greater than about 0.1 m²/g, greater than about 10 m²/g, greater than about 30 m²/g, or greater than about 50 m²/g.

11. The biomedical implant of claim 1, comprising at least about 10 weight percent of the photocatalytically active filler and wherein the photocatalytically active filler comprises crystalline TiO₂ nanoparticles, at least about 50 weight percent of which are of a size less than about 100 nm and wherein at least about 50 weight percent of the TiO₂ nanoparticles of a size less than about 100 nm are of anatase phase.

12. A method for providing an antipathogenic biomedical implant, comprising providing the biomedical implant of claim 1, and irradiating the biomedical implant with light of a wavelength and intensity effective to activate the photocatalytically active filler.

13. The method of claim 12, wherein the biomedical implant comprises at least about 10 weight percent of the photocatalytically active filler and wherein the photocatalytically active filler comprises crystalline TiO₂ nanoparticles, at least about 50 weight percent of which are of a size less than about 100 nm and wherein at least about 50 weight percent of the TiO₂ nanoparticles of a size less than about 100 nm are of anatase phase.

14. A method for reducing pathogens on a biomedical implant, comprising installing the biomedical implant of claim 1 in a patient, wherein the implant is installed in a position such that the photocatalytically active filler is arranged to receive light irradiated from an external source, and irradiating the biomedical implant with light of a wavelength and intensity effective to activate the photocatalytically active filler.

15. The method of claim 14, wherein the biomedical implant is installed in a skin penetrating location in the patient.

16.-17. (canceled)

18. A method for reducing the bioburden in a biomedical implant installation, comprising irradiating light on a biomedical implant comprising at least about 1 weight percent of a photocatalytically active material which exhibits an antipathogenic effect upon irradiation with light, wherein the photocatalytically active material is arranged in the implant to receive light irradiated from an external light source, the irradiating light being of a wavelength and intensity effective to activate the photocatalytically active material, and installing the irradiated implant in a patient.

19. The method of claim 18, wherein the implant is installed in a position such that the photocatalytically active filler is arranged to receive light irradiated from an external source.

20. The method of claim 18, wherein the biomedical implant comprises at least about 10 weight percent of the photocatalytically active material and wherein the photocatalytically active material comprises crystalline TiO₂ nanoparticles, at least about 50 weight percent of which are of a size less than about 100 nm and wherein at least about 50 weight percent of the TiO₂ nanoparticles of a size less than about 100 nm are of anatase phase.

21. A kit for providing an antipathogenic biomedical implant, comprising (a) a biomedical implant comprising at least about 1 weight percent of a photocatalytically active material which exhibits an antipathogenic effect upon irradiation with light, wherein the photocatalytically active material is arranged in the implant to receive light irradiated from an external light source, and (b) a light source operable to emit light of a wavelength and intensity sufficient to cause the photocatalytically active material to exhibit an antipathogenic effect upon irradiation with light from the light source.

22. The kit of claim 21, wherein the biomedical implant comprises at least about 10 weight percent of the photocatalytically active material and wherein the photocatalytically active material comprises crystalline TiO₂ nanoparticles, at least about 50 weight percent of which are of a size less than about 100 nm and wherein at least about 50 weight percent of the TiO₂ nanoparticles of a size less than about 100 nm are of anatase phase.

23. The kit of claim 21, wherein the photocatalytically active material comprises TiO₂ of anatase phase, and wherein the light source emits photons with a wavelength in the range of from 400 nm to 315 nm, preferably in the range of from 385 nm to 315 nm.

24. A kit for providing an antipathogenic biomedical implant, comprising (a) the biomedical implant of claim 1, and (b) a light source operable to emit light of a wavelength and intensity sufficient to cause the photocatalytically active material to exhibit an antipathogenic effect upon irradiation with light from the light source.

25. The kit of claim 24, wherein the photocatalytically active material comprises TiO₂ nanoparticles of anatase phase, and wherein the light source emits photons with a wavelength in the range of from 400 nm to 315 nm, preferably in the range of from 385 nm to 315 nm.
